(19) United States
(12) Patent Application Publication
(10) Pub. No.: US 2026/0346254 A1
(43) Pub. Date: Nov. 5, 2026

(54) DIGITAL PCR DEVICE, DIGITAL PCR EQUIPMENT, AND DIGITAL PCR METHOD

(71) Applicant: HITACHI, LTD., Tokyo (JP)

(72) Inventors: Yoshiki NAKAMURA, Tokyo (JP); Yoshio KAMURA, Tokyo (JP); Tatsuo NAKAGAWA, Tokyo (JP)

(21) Appl. No.: 19/486,178

(22) PCT Filed: Jan. 22, 2024
(86) PCT No.: PCT/JP2024/001630

§ 371 Date: Nov. 20, 2025

(30) Foreign Application Priority Data
Jun. 9, 2023 (JP) 2023-095253

(51) Int. Cl. B01L 7/00

(52) U.S. Cl. CPC B01L 7/52; B01L 2200/026; B01L 2300/041; B01L 2300/12; B01L 2300/1805; B01L 2400/049

(57) ABSTRACT

The present disclosure provides a digital PCR device technology with which it is possible to introduce a solution without leaving air bubbles in a fraction by introducing a sample solution into the fraction by utilizing the air-permeability of a constituent material of a digital PCR device, and which has a high thermal efficiency capable of performing the digital PCR. In the present disclosure, the digital PCR device for performing the digital PCR includes: a chip including a plurality of fractions on a surface, the chip being air-permeable; a lid including an inlet for introducing a solution into the fractions of the chip and an outlet for discharging the solution, the lid covering the chip via a solution chamber; and a support that is fixed to a surface on a side opposite to the lid with respect to the chip, the support having a breathable structure, in which the support has a high thermal conductivity (see FIG. 1).

### TECHNICAL FIELD

The present disclosure relates to a digital PCR device, digital PCR equipment and a digital PCR method.

### BACKGROUND ART

The digital PCR (Polymerase Chain Reaction) is a technology of detecting nucleic acids with high sensitivity, and can detect low-frequency genetic mutations as compared to the conventional real-time quantitative PCR. The measurement flow in the digital PCR includes a process of introducing a nucleic acid solution subjected to limiting dilution into microcompartments on nano-to micro-scales, and a PCR process of controlling the temperature of a solution separated into each compartment.

Patent Literature 1 discloses a device for capturing a nucleic acid for performing gene expression analysis at a single cell level, and describes a method for introducing a sample suspension into a sample capturing section as follows: “After the injection of the cell suspension, a suction operation is performed by sucking with the syringe from the suction port, and the cell suspension is sucked within the chip for capturing a nucleic acid in a single cell.”

Patent Literature 2 discloses a method and an apparatus for simultaneously analyzing the whole genomic expression profile of an organism, and states regarding the thermal efficiency of fractions when controlling the temperature that “the substrate of the microplate assembly comprises a through-hole plate and a backing sheet, wherein the holes in the plate form the sides of the wells and the backing sheet forms the bottom of the wells. The backing sheet comprises a heat conducting material that contacts a thermal cycling device and is capable of transferring the heat to the contents of the reaction chamber.”

### CITATION LIST

### Patent Literature

Patent Literature 1: U.S. Pat. No. 11,299,727 B2
Patent Literature 2: WO 2005/028629 A2

### SUMMARY OF INVENTION

### Technical Problem

Patent Literature 1 describes a method for introducing a sample suspension into microcompartments in a device for capturing a nucleic acid for performing gene expression analysis at a single cell level, by applying a negative pressure from a lower part of the microcompartments in the device into which the sample suspension has been introduced. However, in the device described in Patent Literature 1, when the temperature control required for reactions such as PCR is performed, the temperature is controlled via a material on the underside of the device, a space for applying a negative pressure, and a filter, which results in a problem of decreasing the thermal efficiency. For example, when the device described in Patent Literature 1 is used for the digital PCR, sufficient nucleic acid amplification does not take place due to the thermal efficiency described above, which could fail to quantify the nucleic acid.

Patent Literature 2 describes an apparatus for analyzing the genomic expression profile, which is configured to use a highly heat conducting material for a sheet that forms the bottom of the wells for holding a sample solution. However, in the apparatus described in Patent Literature 2, it is difficult to introduce a sample solution into the wells without leaving air bubbles and division of the solution is failed in performing the digital PCR, which could reduce the quantitative accuracy.

One of the object of the present disclosure is to provide a digital PCR technology with which it is possible to introduce a solution without leaving air bubbles in a fraction by introducing a sample solution into the fraction by utilizing the air-permeability of a constituent material of a device and which has a high thermal efficiency capable of performing the digital PCR.

### Solution to Problem

An example of a digital PCR device according to the present disclosure is a digital PCR device for performing digital PCR, the digital PCR device including: a chip including a plurality of fractions on a surface, the chip being air-permeable; a lid including an inlet for introducing a solution into the fractions of the chip and an outlet for discharging the solution, the lid covering the chip via a solution chamber; and a support that is fixed to a surface on a side opposite to the lid with respect to the chip, the support having a breathable structure, in which the support has a high thermal conductivity.

An example of digital PCR equipment according to the present disclosure is digital PCR equipment for performing digital PCR using the aforementioned digital PCR device, the digital PCR equipment including: a vacuum chamber that fixes the digital PCR device by supporting the support and creates a negative pressure in a space facing a side of the support of the digital PCR device; and a temperature controller that heats the digital PCR device from the side of the support.

An example of digital PCR equipment according to the present disclosure is digital PCR equipment for performing digital PCR using a digital PCR device, in which the digital PCR device includes: a chip including a plurality of fractions on a surface, the chip being air-permeable; and a lid including an inlet for introducing a solution into the fractions of the chip and an outlet for discharging the solution, the lid covering the chip via a solution chamber, and the digital PCR equipment includes: a vacuum chamber including a support having a breathable structure fixed to one surface, the vacuum chamber creating a negative pressure in the one surface; and a temperature controller that heats the digital PCR device from a side of the support, the digital PCR device being disposed on the vacuum chamber.

An example of a digital PCR method according to the present disclosure is a digital PCR method for performing digital PCR using the aforementioned digital PCR device, the method including the steps of: introducing a sample solution through the inlet of the digital PCR device; creating a negative pressure in a space facing a side of the support of the digital PCR device; separating the sample solution by introducing a separation solution through the inlet; and controlling a temperature of the digital PCR device via the support.

### Advantageous Effects of Invention

According to the present disclosure, a high thermal efficiency can be achieved in a device for introducing a sample solution into a fraction by utilizing the air-permeability of the device.

### BRIEF DESCRIPTION OF DRAWINGS

FIG. 1 is a side view showing the configuration of a digital PCR device to which an air-permeable support is fixed.

FIG. 2 is a top view showing the configuration of the digital PCR device shown in FIG. 1.

FIG. 3 is a view illustrating the configuration of equipment in which the digital PCR device shown in FIG. 1 is installed on a vacuum chamber.

FIG. 4 is a view illustrating the configuration of the equipment in which the digital PCR device shown in FIG. 1 is installed on a temperature controller.

FIG. 5 is a view showing a state in which a sample solution is introduced into a solution chamber of the digital PCR device shown in FIG. 1.

FIG. 6 is a view showing a state in which after the sample solution is introduced into the solution chamber of the digital PCR device shown in FIG. 1, a solution is introduced into fractions by a depressurizing operation of the vacuum chamber.

FIG. 7 shows the result of three-dimensional fluorescence observation after a fluorescent solution is introduced into the solution chamber of the digital PCR device shown in FIG. 1, using the method shown in FIG. 5.

FIG. 8 shows the result of three-dimensional fluorescence observation after the fluorescent solution is introduced into the fractions of the digital PCR device shown in FIG. 1, using the method shown in FIG. 6.

FIG. 9 is a view showing a state in which after the sample solution is introduced into the solution chamber and the fractions of the digital PCR device shown in FIG. 1, using the methods shown in FIG. 5 and FIG. 6, a separation solution is introduced into the solution chamber to divide the sample solution into the fractions.

FIG. 10 is a view illustrating a state in which after the sample solution is introduced into the solution chamber and the fractions of the digital PCR device shown in FIG. 1 and the sample solution is further divided into the fractions, using the methods shown in FIG. 5, FIG. 6, and FIG. 9, and then, the digital PCR device is installed on the temperature controller to control the temperature.

FIG. 11 is a fluorescent image showing the result of the digital PCR in Embodiment 1 of the present disclosure.

FIG. 12 is a side view showing the configurations of the digital PCR device and equipment according to Embodiment 2 of the present disclosure.

FIG. 13 is a side view showing the configuration of the digital PCR device according to Embodiment 3 of the present disclosure.

FIG. 14 is a view showing the result of fluorescence measurement after a fluorescent solution is introduced and divided into the fractions of the digital PCR device having the configuration shown in FIG. 13.

FIG. 15 is a view showing the result of the fluorescence measurement after the fluorescent solution is introduced and divided into the fractions of the digital PCR device having the configuration shown in FIG. 13.

FIG. 16 is a side view showing an outline of the configurations of the digital PCR device and equipment according to Embodiment 4 of the present disclosure.

FIG. 17 is a conceptual chart schematically illustrating the result of measurement that can be performed by Embodiment 4 of the present disclosure.

### DESCRIPTION OF EMBODIMENTS

Hereinafter, embodiments of the present disclosure will be described using the drawings.

### Embodiment 1

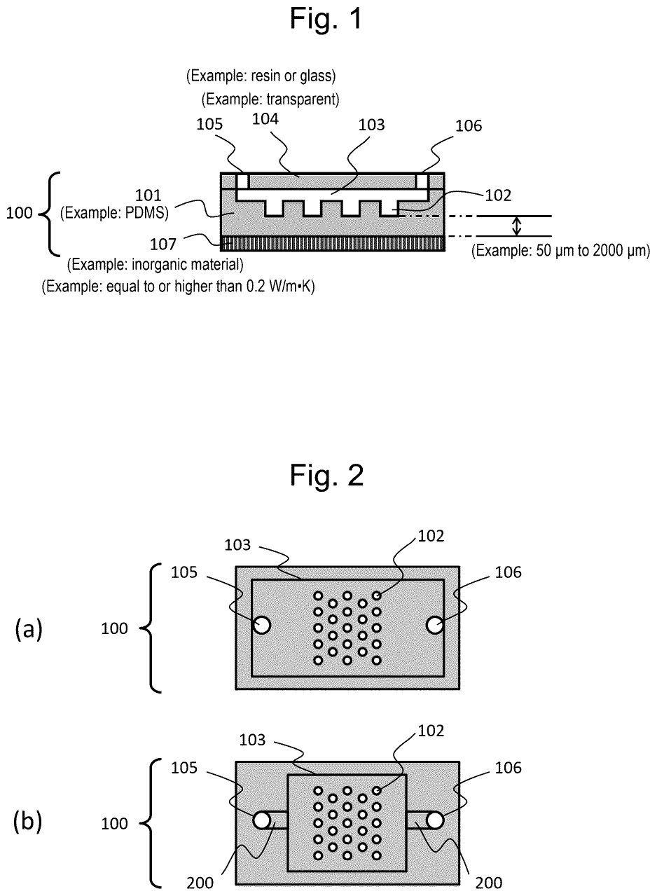

Hereinafter, Embodiment 1 will be described using FIG. 1 to FIG. 11. The present embodiment is a method for performing digital PCR in which in the processes of solution introduction and solution division using a digital PCR device, deformation of the digital PCR device can be suppressed and in the PCR process, the temperature can be controlled with a sufficient thermal efficiency.

FIG. 1 is a side view showing an outline of the configuration of a digital PCR device 100 in the present embodiment. The digital PCR device 100 is used to perform the digital PCR in accordance with a PCR method described in the present specification. The digital PCR device 100 includes an air-permeable chip 101 having a plurality of fractions 102 on its surface.

The material of the chip 101 is preferably, for example, PDMS (poly dimethylsiloxane), which is excellent in air-permeability. The thickness of the chip will be described later.

The number of fractions 102 can be between 1,000 and 100,000,000. The horizontal in-plane shape (hereinafter, a fraction shape) of the fraction 102 may be various shapes, such as a circle, a polygon (a square, a pentagon, a hexagon, or the like), and a combination thereof, and be preferably a circle or a hexagon to secure the planar arrangement density of the fractions.

The width or diameter (hereinafter, a fraction width) of the horizontal in-plane shape of the fraction 102 may be from 1 μm to 1000 μm, and is preferably from 10 μm to 100 μm in terms of the resolution in microscopic observation and the planar arrangement density of the fractions. The depth of the fraction 102 is determined relatively to the fraction width to estimate the volume per fraction depending on the application of the digital PCR device 100, and may be, for example, from 1 μm to 1000 μm.

The planar arrangement periodicity (hereinafter, a fraction periodicity) of the fractions 102 is a value that determines the planar arrangement density of the fractions, and is determined relatively to the fraction width, and may be, for example, from 1.01 to 100 times the fraction width.

The digital PCR device 100 includes a solution chamber 103 on a surface, in which the fractions 102 are formed, of the chip 101. This solution chamber 103 is used to transfer a sample solution and a separation solution. The depth of the solution chamber may be any length that allows the introduction of a liquid by using a syringe, a pneumatic pump, or a pipette operation, and may be from 100 nm to 10 mm.

At least part of the inner surface of the digital PCR device 100, which is composed of the surfaces of the solution chamber 103, the fractions 102, and the chip 101, may be subjected to surface treatment, such as hydrophilization, hydrophobization, and chemical modification, or may not be subjected to any treatment.

The digital PCR device 100 includes a lid 104 that covers the chip 101 via the solution chamber 103 (for example, across the solution chamber 103). The material of the lid 104 is preferably transparent in the visible light spectrum or has a high transmittance to visible light, so as to perform fluorescence detection through the lid. It is also preferable that the material be low in autofluorescence. The thickness of the lid may be from 100 μm to 10 mm to suppress the deformation of the chip.

For the material of the lid 104, for example, resin (acrylic resin, cycloolefin resin, polycarbonate, or the like) and an inorganic material with transparency to visible light (quartz, sapphire, various types of glass, ITO, or the like) may be used. In particular, when the material of the lid 104 is composed of resin or glass, the lid 104 can be easily made transparent.

The lid 104 includes an inlet 105 for introducing a solution into the fractions 102 of the chip 101 and an outlet 106 for discharging the solution. For the method for forming the inlet 105 and the outlet 106, injection molding when the lid is molded, drilling holes in a plate material, and the like may be used.

Here, the thickness of the chip is defined as the sum of the depth of the fractions and the distance from the bottom surface of the fractions to the surface of the chip on a side opposite to the lid (thickness of the chip bottom surface). The thickness of the chip bottom surface (that is, the thickness excluding the depth of the fractions 102 in the chip) is preferably from 50 μm to 2000 μm, because when the thickness is smaller than 50 μm, manufacturing becomes difficult, and when the thickness is greater than 2000 μm, the thermal conductivity and the air-permeability decrease. Note that considering the thermal conductivity of the chip, it is more preferable that the thickness of the chip bottom surface be equal to or smaller than 1000 μm.

The digital PCR device 100 includes a support 107 fixed to the surface of the chip 101 on the side opposite to the lid 104. The support 107 has a breathable structure.

The support 107 has a high thermal conductivity. The thermal conductivity of the support 107 is preferably a value higher than that of the chip material, and may be equal to or higher than 0.2 W/m·K. By setting such a value, the thermal efficiency of the digital PCR device 100 can be improved. Further, the thermal conductivity of the support 107 is preferably equal to or higher than 1 W/m·K, and more preferably, equal to or higher than 100 W/m·K.

Further, since the thermal resistance of the support increases in direct proportion to its thickness, a thinner support is preferred in terms of thermal conductivity, but a certain thickness is required in terms of suppressing the chip deformation. The thickness of the support is, for example, from 10 μm to 5000 μm, and preferably from 50 μm to 500 μm.

The breathable structure has at least one physically continuous through-hole. The material of the support 107 is preferably an inorganic material in order to provide the aforementioned thermal conductivity. For example, gold, silver, copper, platinum, aluminum, tungsten, molybdenum, chromium, titanium, nickel, an alloy containing these as the composition, silicon, silicon carbide, silicon nitride, and glass can be used. By forming the support 107 with an inorganic material, the strength can also be easily secured.

A pore (opening) formed on the surface of the support by the through-hole may be in any shape. For example, various shapes may be used, such as a circle, a polygon (a square, a pentagon, a hexagon, or the like), and a combination thereof.

The width or diameter of the horizontal in-plane shape (hereinafter, a pore width) of the pore may be from 0.1 μm to 1000 μm, and is preferably from 0.1 μm to 100μ in terms of supporting the chip to suppress the deformation. Further, when a porous body is used as the support, the average porosity is preferably equal to or smaller than 90%.

In order to reduce variations in area among the fractions overlapping the surface of the support in the thickness direction, the planar arrangement periodicity (hereinafter, a pore periodicity) of the pores is preferably equal to or smaller than the fraction periodicity, and most preferably, equal to the fraction periodicity. In addition, when the pore periodicity exceeds the fraction periodicity, it is highly likely that variations in thermal efficiency depending on the relative position of the fractions with respect to the pores of the support occur during the temperature control, so that it is preferable to take additional measures. Specifically, the thermal efficiency is insufficient in the fractions overlapping the pores in the thickness direction while the temperature is controlled with a sufficient thermal efficiency in the fractions overlapping the support surface in the thickness direction, which could fail to obtain a sufficient temperature control effect, and therefore, measures to address these issues are preferred to be taken.

FIG. 2 is a top view of an outline of the configuration of the digital PCR device 100. Under the constraint that the constituent members of the digital PCR device 100 are vertically stacked, the illustrations of the lid 104 and the support 107 are omitted. The inlet 105 and the outlet 106 formed in the lid 104 each may be directly connected to the solution chamber 103 as shown in FIG. 2 (a) or indirectly connected to the solution chamber 103 via a flow path 200 as shown in FIG. 2 (b).

The horizontal in-plane shape of the solution chamber 103 may be various shapes, such as a circle, a polygon (a square, a pentagon, a hexagon, or the like), and a combination thereof.

The foregoing is the configuration of the digital PCR device 100 in the present embodiment. Hereinafter, the configuration of a digital PCR equipment in the present embodiment will be described.

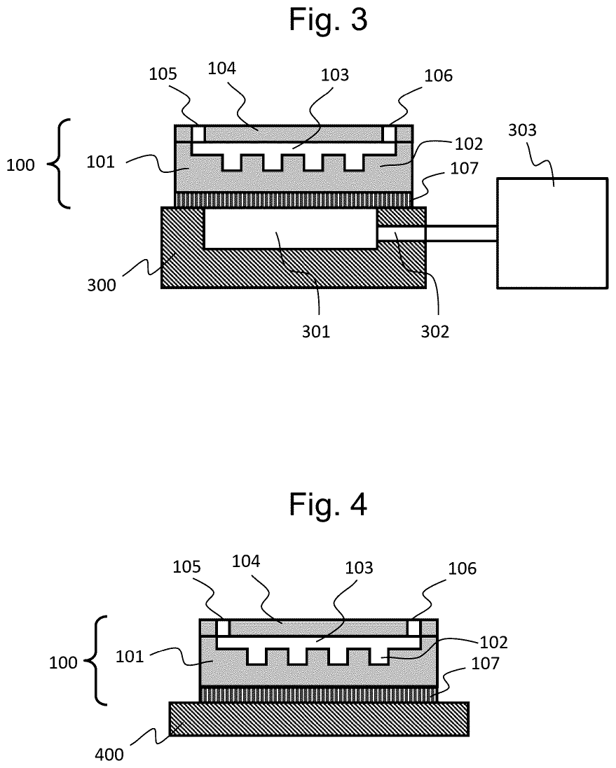

FIG. 3 is a schematic view showing an outline of the configuration of the digital PCR equipment in the processes of solution introduction and solution division using the digital PCR device 100. This digital PCR equipment performs the digital PCR using the digital PCR device 100.

The digital PCR equipment includes a vacuum chamber 300. The vacuum chamber 300 fixes the digital PCR device 100 by supporting the support 107, and creates a negative pressure in a space facing the support 107 side of the digital PCR device 100.

The support 107 in the digital PCR device 100 is disposed so as to cover an open space 301, which is connected to a pressure reducer 303, of the vacuum chamber 300. In this manner, at least part of the surface of the chip 101 faces the open space 301 across the through-holes in the support 107. Therefore, the pressure change in the open space 301 is directly transmitted to the surface of the chip 101.

For example, when the open space 301 is depressurized using the pressure reducer 303 connected via a connection hole 302, the air pressure on the surface of the chip 101 is also reduced, and a pressure difference occurs between the side of the open space 301 and the side of the solution chamber 103 and fractions 102 across the chip 101 and the support 107. Here, by forming the support 107 in the structure having the through-holes as described above, the pressure difference can be appropriately utilized.

Since the chip 101 is composed of an air-permeable material, the air moves across the chip 101 from the side of the solution chamber 103 and fractions 102, which are under relatively high pressure, to the side of the open space 301, which is under relatively low pressure. In other words, it is possible to release the air within the fractions 102 to the outside of the digital PCR device 100 and instead, introduce the sample solution into the fractions 102.

FIG. 4 is a schematic view showing an outline of the configuration of the equipment in the PCR process using the digital PCR device 100. The digital PCR equipment includes a temperature controller 400. The temperature controller 400 heats the digital PCR device 100 from the support 107 side. In the PCR process, after the process of solution division is completed, the digital PCR device 100 is removed from the open space 301 of the vacuum chamber 300 and moved onto the temperature controller 400.

The foregoing is the configuration of the digital PCR equipment in the present embodiment. Hereinafter, the present embodiment will be described following the flow of operation of the present equipment.

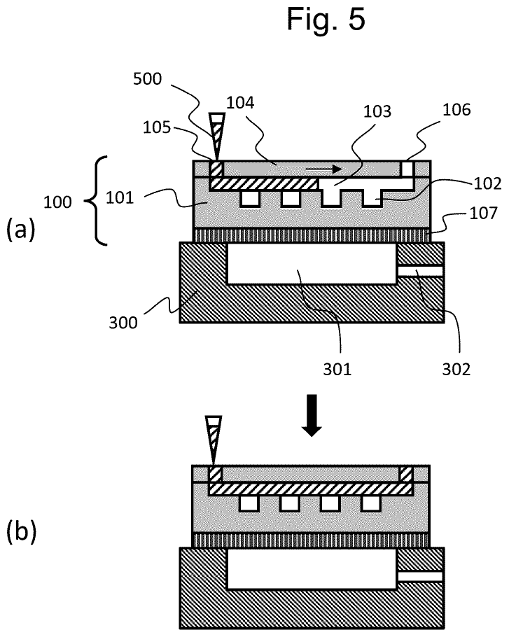

FIG. 5 is a view illustrating the process of introducing a sample solution into the solution chamber 103. A sample solution 500 is connected to the inlet 105 as shown in FIG. 5 (a) and the sample solution 500 or air is pressurized, thereby introducing the sample solution 500 into the solution chamber 103. For the method for applying the pressure, a pipette operation or various pumps such as a syringe pump or a peristaltic pump can be used, and pressurization through the inlet 105 may be performed or depressurization through the outlet 106 may be performed.

Further, for the method for introducing the sample solution 500, the capillary force of the surface characteristics of the inner walls of the solution chamber 103 and the flow path 200 (see FIG. 2) that connects the solution chamber 103 with the inlet 105 and the outlet 106 may be utilized. Through the aforementioned method, the sample solution 500 fills the solution chamber 103 and reaches the outlet 106. Here, if the surfaces of the chip 101, the fractions 102, and the lid 104 that are on the solution chamber 103 side are not subjected to the surface characteristic control, the air in the fractions generally remains without being replaced with the sample solution, as shown in FIG. 5 (b).

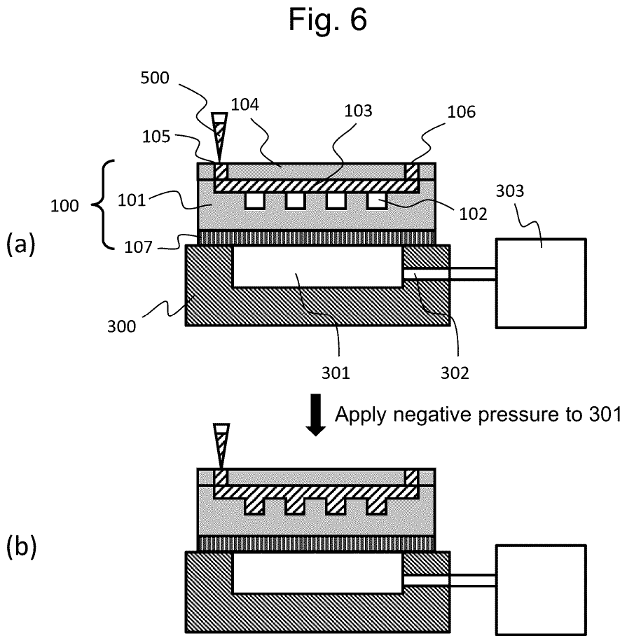

FIG. 6 is a view illustrating the process of introducing the sample solution into the fractions 102. Using the method shown in FIG. 6 (a), the sample solution is introduced into the solution chamber 103 through the inlet 105 of the digital PCR device 100. After introducing the sample solution, the pressure reducer 303 is connected via the connection hole 302 of the vacuum chamber 300, and the open space 301 is depressurized as shown in FIG. 6 (b). That is, a negative pressure is created in the space facing the support 107 side of the digital PCR device 100.

Note that the pressure reducer 303 may be connected to the vacuum chamber 300 either before or after the process of introducing the solution into the solution chamber 103. With this operation, the process of introducing the sample solution into the solution chamber 103 and the fractions 102 is completed.

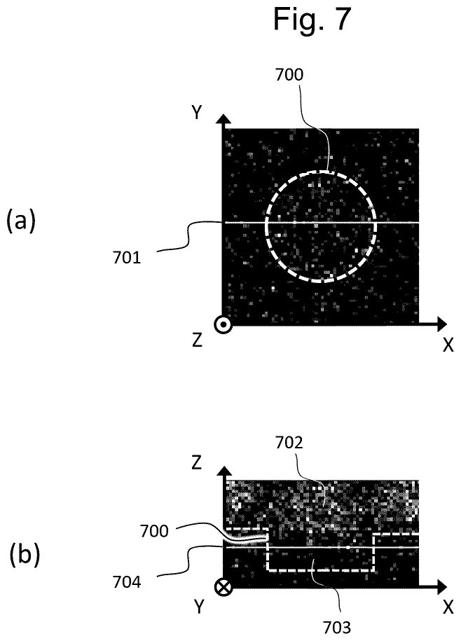

FIG. 7 shows the result of three-dimensional fluorescence imaging performed after introducing, using the method illustrated in FIG. 5, a fluorescent solution into the solution chamber of the digital PCR device 100 in which the inner surface is not subjected to the surface treatment and which has circular fractions. FIG. 7 (a) is a fluorescent image in an X-Y cross section 704 (illustrated in FIG. 7 (b)) passing through the fraction at the central depth, and FIG. 7 (b) is a fluorescent image in an X-Z cross section 701 (illustrated in FIG. 7 (a)) passing through the fraction at the center. Further, a fraction wall surface 700 is denoted by a dashed line.

In FIG. 7 (a), no region with a high fluorescence intensity is observed. This indicates that the fluorescent solution is not introduced into the fraction. In FIG. 7 (b), the fluorescence intensity can be observed in the region of a solution chamber 702, but not in the region of a fraction interior portion 703. This indicates that the fluorescent solution is introduced into the solution chamber, but not into the fraction.

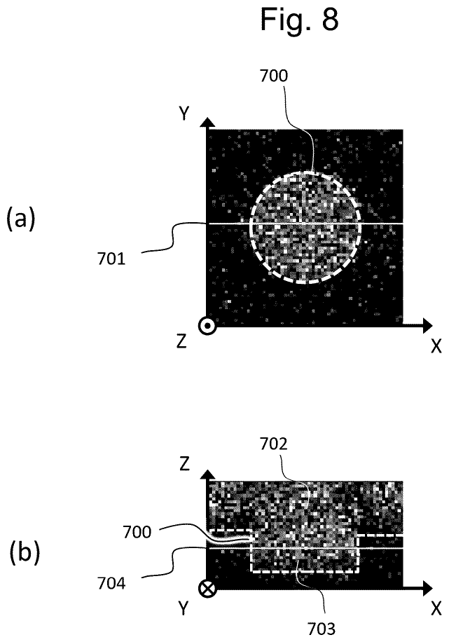

FIG. 8 shows the result of three-dimensional fluorescence imaging performed after introducing, using the method illustrated in FIG. 5, a fluorescent solution into the solution chamber of the digital PCR device 100 in which the inner surface is not subjected to the surface treatment and which has circular fractions, and then introducing, using the method illustrated in FIG. 6, the fluorescent solution into the fraction. FIG. 8 (a) is a fluorescent image in the X-Y cross section 704 (illustrated in FIG. 8 (b)) passing through the fraction at the central depth, and FIG. 8 (b) is a fluorescent image in the X-Z cross section 701 (illustrated in FIG. 8 (a)) passing through the fraction at the center. Further, the fraction wall surface 700 is denoted by a dashed line.

In FIG. 8 (a), a high fluorescence intensity is observed on the inner side of the fraction wall surface 700. This indicates that the fluorescent solution is introduced into the fraction at least up to the middle height. In FIG. 8 (b), a high fluorescence intensity can also be observed in the region of the fraction interior portion 703 as well as the solution chamber 702. In addition, the fluorescence intensity can be observed all the way to the bottom of the fraction. This indicates that the fluorescent solution is introduced into the solution chamber and the fraction.

The experiment results in FIG. 7 and FIG. 8 have confirmed that the method for introducing a solution in the present embodiment enables the solution introduction into the fractions without performing the surface treatment on the inner surface of the digital PCR device 100.

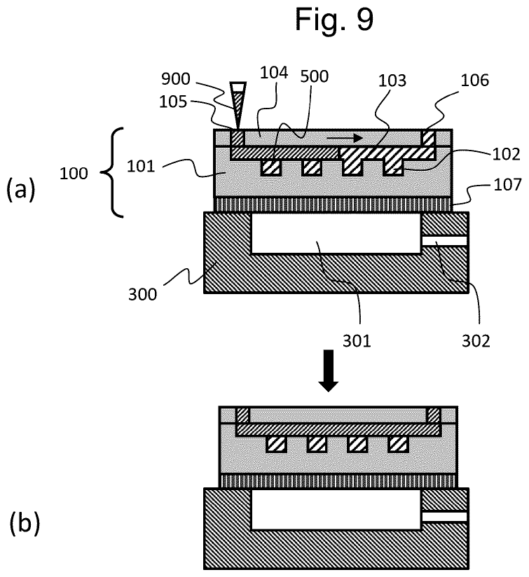

FIG. 9 is a view illustrating the process of dividing a sample solution into the fractions 102. After the process of introducing, using the methods illustrated in FIG. 5 and FIG. 6, the sample solution into the solution chamber 103 and the fractions 102 is completed, a separation solution 900 is connected to the inlet 105 as shown in FIG. 9 (a) and the separation solution or air is pressurized, thereby introducing the separation solution into the solution chamber 103 as shown in FIG. 9 (b).

Here, the separation solution is a liquid that is immiscible with water, and for example, mineral oil, silicone oil, and fluorine-based oil may be used. For the method for applying the pressure to the separation solution, a pipette operation or various pumps such as a syringe pump or a peristaltic pump can be used, and pressurization through the inlet 105 may be performed or depressurization through the outlet 106 may be performed.

With this operation, the separation solution 900 fills the solution chamber 103 and reaches the outlet 106. At this time, it is expected that introducing the separation solution into the solution chamber 103 removes the sample solution from the surface of the chip 101. In this manner, the sample solution is divided into the fractions and lose fluid continuity. That is, the sample solution can be separated by introducing the separation solution through the inlet 105.

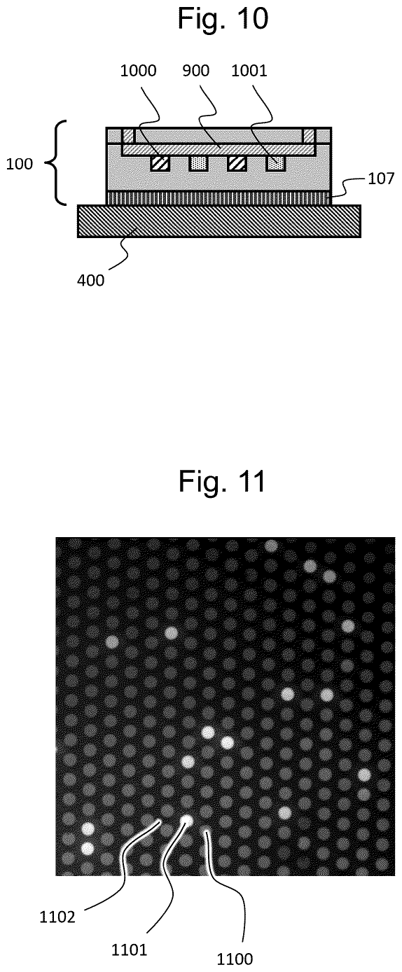

FIG. 10 is a view illustrating the PCR process. After the process of solution division is completed, the digital PCR device 100 is removed from the open space 301 of the vacuum chamber 300 and moved onto the temperature controller 400 as in the configuration shown in FIG. 4. With this configuration, the temperature of the digital PCR device 100 is controlled via the support 107 using the temperature controller, and the PCR is performed on the sample solution. At this time, the sample solution divided into the fractions 102 of the digital PCR device 100 is heated and cooled by the temperature controller via the chip 101 and the support. The nucleic acid amplification reaction does not occur in a digital PCR negative fraction 1000 while the nucleic acids are amplified in a digital PCR positive fraction 1001.

The foregoing is the operation of the digital PCR equipment in the present embodiment. After the PCR process, in the digital PCR device 100, fluorescence detection is performed through the lid 104. The method for the fluorescence detection can be appropriately designed by a person skilled in the art based on the publicly-known techniques or the like, and thus will be omitted.

FIG. 11 shows the results of fluorescence observation after the digital PCR according to the present embodiment. A clear difference in fluorescence intensity between a digital PCR negative fraction 1100 and a digital PCR positive fraction 1101 can be observed. Further, a region with a distinctly high fluorescence intensity is not observed in a non-fraction area 1102. This indicates that the state of the solution divided into the fractions is stably retained in the PCR process. These results indicate that the present embodiment can properly perform the digital PCR.

The digital PCR device 100 of the present embodiment includes the chip 101 having the plurality of fractions 102 on its surface and composed of an air-permeable material, the solution chamber 103, the lid including the inlet 105 and the outlet 106 and covering the solution chamber 103, and the support 107 having a high thermal conductivity and a breathable structure.

The chip deformation causes the divided solution to splash out to the solution chamber or to combine with the solution in the adjacent fraction, resulting in a reduced determination accuracy of the digital PCR. In the present embodiment, since the support is sufficiently rigid relative to the chip, even when a thin chip is used, the chip deformation can be suppressed throughout the processes of solution introduction, solution division, and PCR.

There is a concerned that with the support inserted between the chip and the temperature controller, the efficiency of the temperature control of the solution in the fractions could be reduced, but with the use of a material with a high thermal conductivity for the support, the digital PCR that requires a high thermal efficiency can be performed.

Use of the air-permeability of the chip material for introducing the solution into the fractions eliminates the need for performing the surface treatment such as hydrophilization, hydrophobization, or chemical modification on the inner surface of the digital PCR device 100, which enables cost reduction. Further, since the solution can be introduced into the fractions under any conditions of the inflow rate in the solution introduction, the solution viscosity, and the wettability of the solution with respect to the inner surface of the digital PCR device 100, the percentage of the fractions where the solution introduction can be properly completed increases.

### Embodiment 2

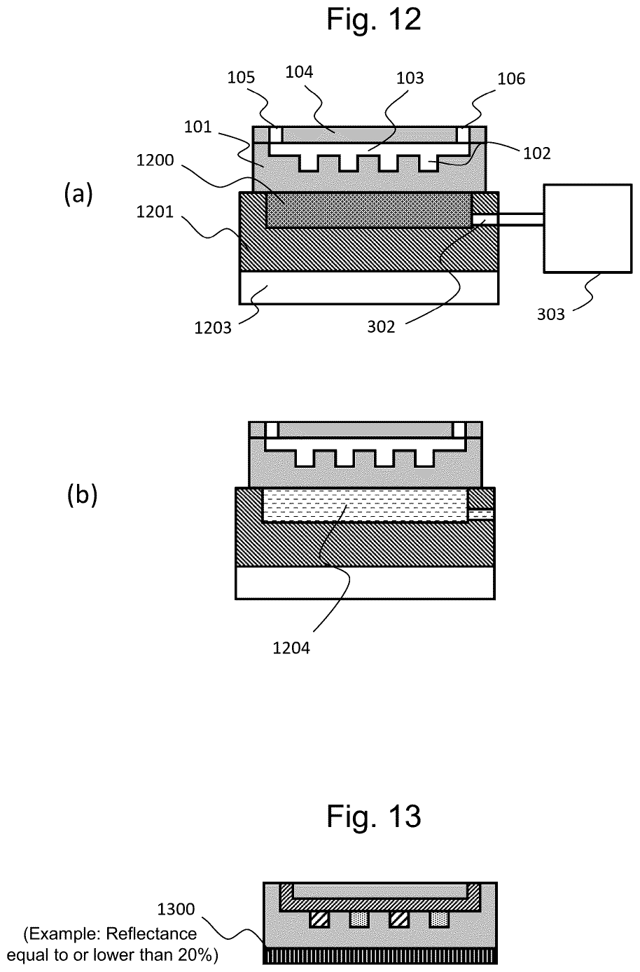

Next, Embodiment 2 will be described using FIG. 12. In Embodiment 2, the support 107 of Embodiment 1 is fixed to the open space 301 of the vacuum chamber instead of being fixed to the digital PCR device, thereby achieving a simplified implementation process and device cost reduction. Note that in FIG. 12, since the same reference numerals as those in FIG. 1 to FIG. 11 denote the same components, the repeated description will be omitted.

FIG. 12 (a) is a schematic view of the configuration of the digital PCR equipment of Embodiment 2. The configuration of the digital PCR device of Embodiment 2 differs from that of Embodiment 1 only in that it does not have a support. In Embodiment 2, a support 1200 having a breathable structure is directly embedded in an opening of a vacuum chamber 1201. The vacuum chamber 1201 has the support 1200 fixed to one surface, and the vacuum chamber 1201 can create a negative pressure on this one surface. Further, in Embodiment 2, the digital PCR device is fixed to a surface, which has the support 1200, of the vacuum chamber 1201.

The digital PCR equipment includes a temperature controller 1203. The temperature controller 1203 heats, from the support 1200 side, the digital PCR device disposed on the vacuum chamber 1201. Further, the vacuum chamber 1201 is fixed to the temperature controller 1203.

In the PCR process of Embodiment 2, the sample solution in the fractions is heated and cooled by the temperature controller 1203 via the chip 101, the support 1200, and the vacuum chamber 1201. Therefore, in Embodiment 2, it is preferable that the vacuum chamber 1201 as well as the support 1200 be composed of a material with a high thermal conductivity. As the material of the vacuum chamber, for example, metal (gold, silver, copper, platinum, aluminum, tungsten, molybdenum, chromium, titanium, nickel, an alloy containing these metals as the composition, or the like) or a semiconductor (silicon, silicon carbide, silicon nitride, or the like) can be used.

As shown in FIG. 12 (b), in the PCR process of Embodiment 2, the vacuum chamber and the interior of the breathable structure of the support may be filled with a liquid 1204. This improves the thermal efficiency of the entire equipment, thereby facilitating the heat transfer from the temperature controller to the fractions in the digital PCR device. The liquid to be used for the filling may be, for example, water, silicone oil, mineral oil, liquid paraffin, or alcohol, but is not limited thereto. Further, a filler may be dispersed in order to improve the thermal conductivity of the liquid to be used for the filling.

The foregoing is the configuration of the digital PCR equipment in the present embodiment. Note that the operation flow of the present equipment is basically the same as that of Embodiment 1, and therefore, the description thereof will be omitted.

The configuration of the equipment of Embodiment 2 differs from that of Embodiment 1 particularly in that the support 1200 is fixed to the vacuum chamber 1201 instead of the digital PCR device and in that a high thermal conductivity is preferred in the vacuum chamber 1201.

The need for moving the digital PCR device throughout the entire digital PCR workflow including the solution introduction, the solution division, and the PCR, which enables stable solution division, simplified implementation process, and device cost reduction. Further, an optical system for performing the fluorescence detection in the digital PCR is also incorporated into the configuration of the equipment of the present embodiment, so that the need for moving the digital PCR device is eliminated between installation of the digital PCR device and the digital PCR detection. This is an advantageous feature in automating the digital PCR workflow.

### Embodiment 3

Next, Embodiment 3 will be described using FIG. 13 to FIG. 15. Embodiment 3 is configured with the support of Embodiment 1 provided with an anti-reflection mechanism, which enables to reduce background light intensity and variations in background light intensity in the fluorescence detection.

FIG. 13 is a schematic view illustrating the configuration of the digital PCR device of Embodiment 3. The configuration of the digital PCR device of Embodiment 3 differs from that of Embodiment 1 only in that a support 1300 has an anti-reflection mechanism.

For the support 1300, a material having a breathable structure and a low reflectance with respect to visible light can be used. For example, gas-permeable resin (composed of one or more of polyester, polyethylene, polypropylene, polystyrene, nylon, polyurethane, cellulose, cycloolefin, epoxy, and vinyl chloride), gas-permeable metal (composed of gold, silver, copper, platinum, aluminum, tungsten, molybdenum, chromium, titanium, nickel, alloys containing these as the composition, and the like), gas-permeable non-metal (composed of one or more of silicon, silicon carbide, silicon nitride, alumina, glass, and carbon), composites thereof, and the like can be used. Further, these anti-reflection layers may be subjected to secondary processing such as surface treatment or coating, or filled with fillers, for the purpose of reducing reflected light.

In particular, when the reflectance of the support 1300 in the visible light spectrum is low, in the fluorescence detection, the light reflection toward the upper side of the digital PCR device can be prevented, thereby enabling to reduce background light intensity and variations in background light intensity in the fluorescence detection. For example, the reflectance in the visible light spectrum is preferably equal to or lower than 20%.

Further, considering the efficiency of the temperature control of the sample solution in the fractions, the thermal conductivity of the anti-reflection layer is preferably equal to or higher than 1 W/m·K.

The foregoing is the configuration of the digital PCR equipment in the present embodiment. Note that the operation flow of the present equipment is basically the same as that of Embodiment 1, and therefore, the description thereof will be omitted.

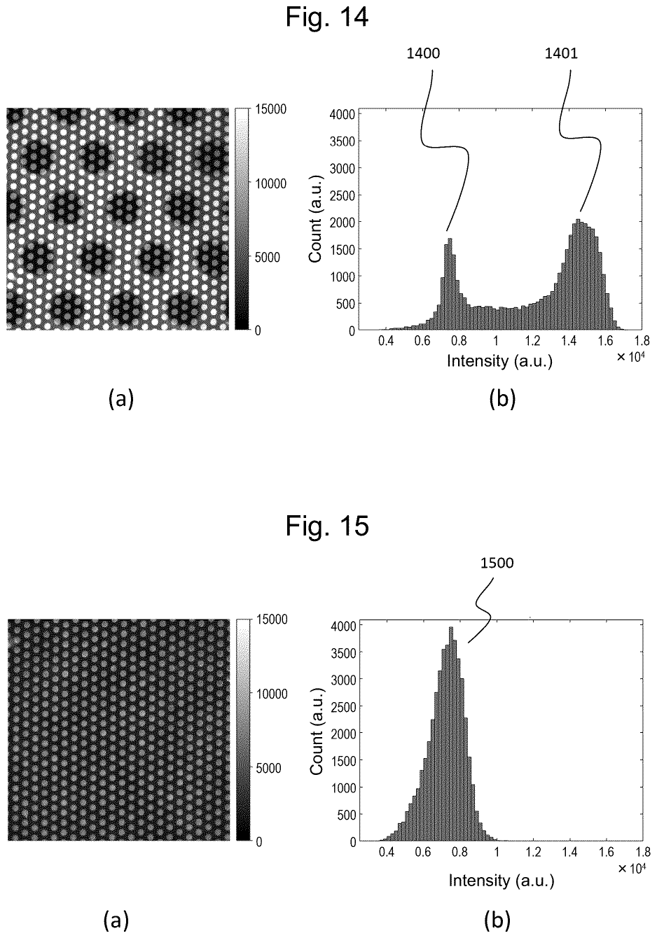

FIG. 14 shows the result of fluorescence measurement performed after dividing a fluorescent solution in the digital PCR device using a PDMS chip having a thickness of 0.3 mm including a circular fraction having a diameter of 0.1 mm, a depth of 0.1 mm, and a fraction periodicity of 0.15 mm, in a case where as the support, an aluminum alloy plate having a thickness of 0.5 mm including a circular through-hole having a diameter of 0.5 mm and a pore periodicity of 1 mm was used.

FIG. 14 (a) is a fluorescent image of the digital PCR device in which the fluorescent solution is divided into the fractions, and FIG. 14 (b) is a histogram of the fluorescent intensity averaged for each fraction.

It can be observed from FIG. 14 (a) that a fluorescent molecule aqueous solution is divided, since the intensity is high on the inner side of the fraction and low on the outer side. However, regions with a high fluorescence intensity are also observed, following the shape of the support surface. This is because an excitation light was reflected by the support and was not fully filtered out by a fluorescence filter for fluorescence measurement, resulting in appearing on the fluorescent image. The magnitude relationship between the reflected light intensity and the fluorescence intensity in the well can be read from FIG. 14 (a). That is, a first peak 1400 (intensity: approximately 7500) in FIG. 14 (b) indicates the fluorescence intensity in the fraction accommodated on the inner side of the through-hole of the support, while a second peak 1401 (intensity: approximately 15000) indicates a value of the fluorescence intensity in the fraction to which the intensity of the light reflected by the support is added.

These results suggest that when an aluminum alloy plate is used for the support, the reflected light at a level equivalent to that of the fluorescence intensity in the fractions is superimposed on the fluorescent image in the fractions in the region overlapping the surface of the support, so that the background intensity increases, resulting in reduction in the measurement accuracy. Further, when the pore periodicity of the support is greater than the fraction periodicity, a clear difference occurs in fluorescence intensity between the fractions overlapping and those not overlapping the pores in the thickness direction, which could result in variations in fluorescence intensity between the fractions and hinder the determination of positivity and negativity in the digital PCR.

FIG. 15 shows the result of fluorescence measurement performed in a case where a copper fiber/resin fiber composite was used as the anti-reflection layer to be inserted between the support and the chip in Embodiment 3. The conditions for the digital PCR device, sample solution introduction, solution separation, and fluorescence measurement differ from those in FIG. 14 only in that the anti-reflection layer is inserted at the time of the fluorescence measurement.

In FIG. 15 (a), variations in fluorescence intensity following the shape of the support surface are not observed. Similarly, also in FIG. 15 (b), variations in fluorescence intensity between the fractions are not observed. Further, since the peak intensity of a peak 1500 in FIG. 15 (b) is approximately 7500, which roughly coincides with the position of the first peak in FIG. 14 (b), it is suggested that by inserting a metal fiber/resin fiber composite as the anti-reflection layer, the influence of the light reflected by the support can be eliminated.

The configuration of the equipment of Embodiment 3 differs from that of Embodiment 1 particularly in that the anti-reflection layer is inserted between the chip and the support.

In the fluorescence detection, the anti-reflection layer prevents the excitation light from reaching the support surface and prevents the light reflection toward the upper side of the digital PCR device, thereby enabling to reduce background light intensity and variations in background light intensity in the fluorescence detection.

### Embodiment 4

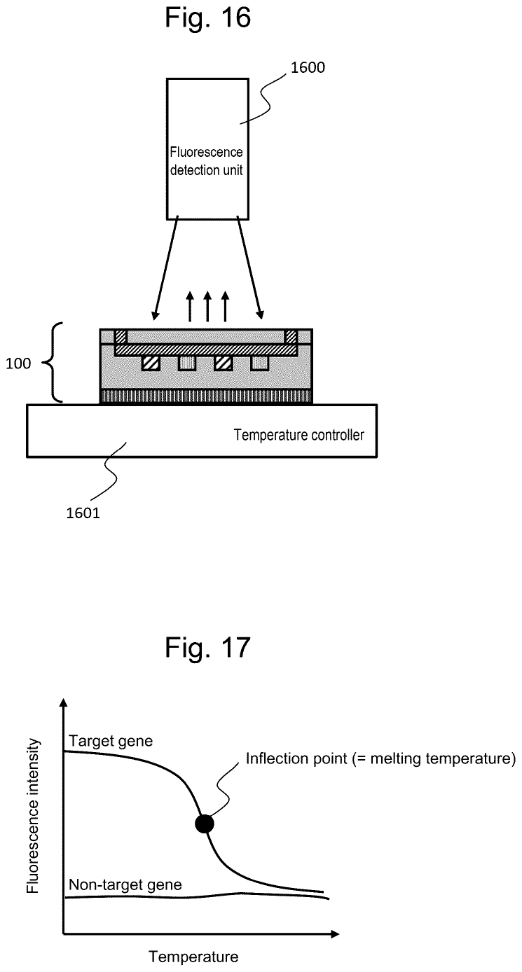

Next, Embodiment 4 will be described using FIG. 16 and FIG. 17. Embodiment 4 relates to a method for performing fluorescence measurement while controlling the temperature of the digital PCR device using the temperature controller in the methods or configurations of Embodiments 1 to 3.

FIG. 16 is a schematic view illustrating an outline of the configuration of the equipment for performing fluorescence measurement while controlling the temperature of the digital PCR device in Embodiment 4. In the digital PCR device 100, the fractions are held on the support side relative to the solution chamber. Thus, when the temperature is controlled from the lid side in the PCR process, the heat is transferred to the solution via the lid and the solution chamber filled with a separation solution, resulting in poor thermal efficiency, and the temperature of the solution does not follow the temperature of the temperature controller and the nucleic acid amplification does not take place.

Accordingly, in order to simultaneously perform the temperature control and the fluorescence measurement using the digital PCR device of Embodiments 1 to 3, it is preferable to install a temperature controller 1601 on the support side to control the temperature and to install a fluorescence detection unit 1600 on the lid side to perform the fluorescence measurement from the lid side, as shown in FIG. 16. The fluorescence detection unit 1600 includes a light source, an excitation filter, a fluorescence filter, a mirror, a dichroic mirror, a lens, a detector, a spectrometer, and the like. With the use of the configuration of the equipment of FIG. 16, it is possible to control the temperature using the temperature controller 1601 and simultaneously perform any fluorescence measurement.

FIG. 17 is a schematic chart showing an example of a melting curve analysis that can be performed by Embodiment 4. The melting curve analysis is a technique of determining the type of a target gene by measuring and analyzing the melting curve using the target gene in a sample and a fluorescently labeled probe that hybridizes to the target gene. To measure the melting curve, the temperature dependency of the fluorescence intensity of the fluorescently labeled probe is measured.

In the configuration of the equipment of Embodiment 4, after performing the PCR on a sample solution containing a fluorescently-labeled probe for the melting curve analysis, the fluorescence measurement is performed while controlling the temperature of the digital PCR device, so as to measure the melting curve of the target gene. By analyzing an inflection point of the melting curve, it is possible to obtain the melting temperature dependent on the target gene. That is, the melting curve analysis can be performed in the present embodiment.

The configuration of the equipment of Embodiment 4 is composed of the temperature controller, the digital PCR device, and the fluorescence detection unit, which are arranged in this order in any direction. In the present embodiment, with this configuration of the equipment, the temperature control and the fluorescence detection are simultaneously performed.

The melting curve analysis can be performed by performing the fluorescence measurement while controlling the temperature.

Note that the present disclosure is not limited to the aforementioned embodiments, and includes various modifications. For example, the aforementioned embodiments have been described in detail to facilitate understanding of the present disclosure, and are not necessarily limited to those including all the described configurations. Further, it is possible to replace part of the configuration of one embodiment with the configuration of another embodiment, and it is also possible to add the configuration of one embodiment to the configuration of another embodiment. Furthermore, for part of the configuration of each embodiment, addition and deletion of and replacement with another configuration are possible.

### REFERENCE SIGNS LIST

100 Digital PCR device
101 Chip
102 Fraction
103 Solution chamber
104 Lid
105 Inlet
106 Outlet
107 Support
200 Flow path
300 Vacuum chamber
301 Open space
302 Connection hole
303 Pressure reducer
400 Temperature controller
500 Sample solution
700 Fraction wall surface
70 X-Z cross section passing through fraction at center
702 Solution chamber
703 Fraction interior portion
704 X-Y cross section passing through fraction at central height
900 Separation solution
1000 Digital PCR negative fraction
1001 Digital PCR positive fraction
1100 Digital PCR negative fraction
1101 Digital PCR positive fraction
1102 Non-fraction area
1200 Support
1201 Vacuum chamber
1203 Temperature controller
1204 Liquid
1300 Support
1400 First peak
1401 Second peak
1500 Peak
1600 Fluorescence detection unit
1601 Temperature controller

1. A digital PCR device for performing digital PCR, the digital PCR device comprising:
a chip including a plurality of fractions on a surface, the chip being air-permeable;
a lid including an inlet for introducing a solution into the fractions of the chip and an outlet for discharging the solution, the lid covering the chip via a solution chamber; and
a support that is fixed to a surface of the chip on a side opposite to the lid, the support having a breathable structure,
wherein the support has a high thermal conductivity.

2. The digital PCR device according to claim 1, wherein a material of the chip is PDMS.

3. The digital PCR device according to claim 1, wherein a thickness of the chip excluding a depth of the fractions is 50 μm to 2000 μm.

4. The digital PCR device according to claim 1, wherein the lid is composed of a material that is transparent in a visible light spectrum.

5. The digital PCR device according to claim 4, wherein the material of the lid is composed of resin or glass.

6. The digital PCR device according to claim 1, wherein the support is composed of an inorganic material.

7. The digital PCR device according to claim 1, wherein the breathable structure has at least one through-hole.

8. The digital PCR device according to claim 1, wherein the support has a low reflectance in a visible light spectrum.

9. The digital PCR device according to claim 8, wherein the reflectance is equal to or lower than 20%.

10. The digital PCR device according to claim 1, wherein the thermal conductivity of the support is equal to or higher than 0.2 W/(m·K).

11. Digital PCR equipment for performing digital PCR using the digital PCR device according to claim 1, the digital PCR equipment comprising:
a vacuum chamber that fixes the digital PCR device by supporting the support and creates a negative pressure in a space facing a side of the support of the digital PCR device; and
a temperature controller that heats the digital PCR device from the side of the support.

12. Digital PCR equipment for performing digital PCR using a digital PCR device, wherein
the digital PCR device comprises:
a chip including a plurality of fractions on a surface, the chip being air-permeable; and
a lid including an inlet for introducing a solution into the fractions of the chip and an outlet for discharging the solution, the lid covering the chip via a solution chamber, and
the digital PCR equipment comprises:
a vacuum chamber including a support having a breathable structure fixed to one surface, the vacuum chamber creating a negative pressure in the one surface; and
a temperature controller that heats the digital PCR device from a side of the support, the digital PCR device being disposed on the vacuum chamber.

13. A method for performing digital PCR using the digital PCR device according to claim 1, the method comprising the steps of:
introducing a sample solution through the inlet of the digital PCR device;
creating a negative pressure in a space facing a side of the support of the digital PCR device;
separating the sample solution by introducing a separation solution through the inlet; and
controlling a temperature of the digital PCR device via the support.
